SEQUENCE LISTING (12 sequences)

SEQ ID NO: 1 - DNA, 63 bp, Artificial Sequence
  Description of Artificial Sequence Primer
ggccagtgaattgtaatacgactcactatagggaggcggtttttttttttttttttttttttt

SEQ ID NO: 2 - DNA, 25 bp, Artificial Sequence
  Description of Artificial Sequence Primer
gtcgtcaagatgctaccgttcagga

SEQ ID NO: 3 - DNA, 1819 bp, Homo sapiens
tcagccaatcacagtttgaaacctttgccctctgttccagaagagaaaaagcccaggcataaaatcatctccatattctcaggcacagagaaaggaagtaaaaagaaagaaaaggaacggccagaaatttctcctccatctgattttgagcacaccatccatgttggctttgatactgttactggagaattcactggcatgccagaacagtgggctcgattactacagacctccaatatcaccaaactagagcaaaagaagaatcctcaggctgtgctggatgtcctaaagttctacgactccaacacagtgaagcagaaatatctgagctttactcctcctgagaaagatggctttccttctggaacaccagcactgaatgccaagggaacagaagcacccgcagtagtgacagaggaggaggatgatgatgaagagactgctcctcccgttattgccccgcgaccggatcatacaaaatcaatttacacacggtctgtaattgaccctgttcctgcaccagttggtgattcacatgttgatggtgctgccaagtctttagacaaacagaaaaagaagactaagatgacagatgaagagattatggagaaattaagaactatcgtgagcataggtgaccctaagaaaaaatatacaagatatgaaaaaattggacaaggggcttctggtacagttttcactgctactgacgttgcactgggacaggaggttgctatcaaacaaattaatttacagaaacagccaaagaaggaactgatcattaacgagattctggtgatgaaagaattgaaaaatcccaacatcgttaactttttggacagttacctggtaggagatgaattgtttgtggtcatggaataccttgctgggaggtcactcactgatgtggtaacagaaacgtgcatggatgaagcacagattgctgctgtatgcagagagtgtttacaggcattggagtttttacatgctaatcaagtgatccacagagacatcaaaagtgacaatgtacttttgggaatggaaggatctgttaagctcactgactttggtttctgtgcccagatcacccctgagcagagcaaacgcagtaccatggtcggaacgccatactggatggcaccagaggtggttacacggaaagcttatggccctaaagtcgacatatggtctctgggtatcatggctattgagatggtagaaggagagcctccatacctcaatgaaaatccccttagggccttgtacctaatagcaactaatggaaccccagaacttcagaatccagagaaactttccccaatatttcgggatttcttaaatcgatgtttggaaatggatgtggaaaaaaggggttcagccaaagaattattacagcatcctttcctgaaactggccaaaccgttatctagcttgacaccactgatcatggcagctaaagaagcaatgaagagtaaccgttaacatcactgctgtggcctcatactcttttttccattttctacaagaagccttttagtatatgaaaattattactctttttggggtttaaagaaatggtctgcataacctgaatgaaagaagcaaatgactattctctgaagacaaccaagagaaaattgcaaaaagacaagtatgacttttatatgaaccccttctttagggtccagaaggaattgtggactgaatcactagccttaggtctttcagcaaacagcctatcagggccatttatcatgtgtgagatttgcattttactttgctgactttgttgtaatagatcccattcattgtccccttt

SEQ ID NO: 4 - protein, 524 aa, Homo sapiens
MSDNGELEDKPPAPPVRMSSTIFSTGGKDPLSANHSLKPLPSVPEEKKPRHKIISIFSGTEKGSKKKEKERPEISPPSDFEHTIHVGFDAVTGEFTGMPEQWARLLQTSNITKLEQKKNPQAVLDVLKFYDSNTVKQKYLSFTPPEKDGLPSGTPALNAKGTEAPAVVTEEEDDDEETAPPVIAPRPDHTKSIYTRSVIDPVPAPVGDSHVDGAAKSLDKQKKKPKMTDEEIMEKLRTIVSIGDPKKKYTRYEKIGQGASGTVFTATDVALGQEVAIKQINLQKQPKKELIINEILVMKELKNPNIVNFLDSYLVGDELFVVMEYLAGGSLTDVVTETCMDEAQIAAVCRECLQALEFLHANQVIHRDIKSDNVLLGMEGSVKLTDFGFCAQITPEQSKRSTMVGTPYWMAPEVVTRKAYGPKVDIWSLGIMAIEMVEGEPPYLNENPLRALYLIATNGTPELQNPEKLSPIFRDFLNRCLEMDVEKRGSAKELLQHPFLKLAKPLSSLTPLIMAAKEAMKSNR

SEQ ID NO: 5 - DNA, 50 bp, Artificial Sequence
  Description of Artificial Sequence Primer
ggggacaagtttgtacaaaaaagcaggctatgtctgataacggagaactg

SEQ ID NO: 6 - DNA, 53 bp, Artificial Sequence
  Description of Artificial Sequence Primer
ggggaccactttgtacaagaaagctgggtttaacggttactcttcattgcttc

SEQ ID NO: 7 - DNA, 31 bp, Artificial Sequence
  Description of Artificial Sequence Primer
aagaattctcatgtctgataacggagaactg

SEQ ID NO: 8 - DNA, 29 bp, Artificial Sequence
  Description of Artificial Sequence Primer
tttctagaacggttactcttcattgcttc

SEQ ID NO: 9 - DNA, 3255 bp, Homo sapiens
ggagcgcaaatggcgtccaaccccgaacggggggagattctgctcacggaactgcagggggattcccgaagtcttccgttttctgagaatgtgagtgctgttcaaaaattagacttttcagatacaatggtgcagcagaaattggatgatatcaaggatcgaattaagagagaaataaggaaagaactgaaaatcaaagaaggagctgaaaatctgaggaaagtcacaacagataaaaaaagtttggcttatgtagacaacattttgaaaaaatcaaataaaaaattagaagaactacatcacaagctgcaggaattaaatgcacatattgttgtatcagatccagaagatattacagattgcccaaggactccagatactccaaataatgaccctcgttgttctactagcaacaatagattgaaggccttacaaaaacaattggatatagaacttaaagtaaaacaaggtgcagagaatatgatacagatgtattcaaatggatcttcaaaggatcggaaactccatggtacagctcagcaactgctccaggacagcaagacaaaaatagaagtcatacgaatgcagattcttcaggcagtccagactaatgaattggcttttgataatgcaaaacctgtgataagtcctcttgaacttcggatggaagaattaaggcatcattttaggatagagtttgcagtagcagaaggtgcaaagaatgtaatgaaattacttggctcaggaaaagtaacagacagaaaagcactttcagaagctcaagcaagatttaatgaatcaagtcagaagttggaccttttaaagtattcattagagcaaagattaaacgaagtccccaagaatcatcccaaaagcaggattattattgaagaactttcacttgttgctgcatcaccaacactaagtccacgtcaaagtatgatatctacgcaaaatcaatatagtacactatccaaaccagcagcactaacaggtactttggaagttcgtcttatgggctgccaagatatcctagagaatgtccctggacggtcaaaagcaacatcagttgcactgcctggttggagtccaagtgaaaccagatcatctttcatgagcagaacgagtaaaagtaaaagcggaagtagtcgaaatcttctaaaaaccgatgacttgtccaatgatgtctgtgctgttttgaagctcgataatactgtggttggccaaactagctggaaacccatttccaatcagtcatgggaccagaagtttacactggaactggacaggtcacgtgaactggaaatttcagtttattggcgtgattggcggtctctgtgtgctgtaaaatttctgaggttagaagattttttagacaaccaacggcatggcatgtgtctctatttggaaccacagggtactttatttgcagaggttaccttttttaatccagttattgaaagaagaccaaaacttcaaagacaaaagaaaattttttcaaagcaacaaggcaaaacatttctcagagctcctcaaatgaatattaatattgccacttggggaaggctagtaagaagagctattcctacagtaaatcattctggcaccttcagccctcaagctcctgtgcctactacagtgccagtggttgatgtacgcatccctcaactagcacctccagctagtgattctacagtaaccaaattggactttgatcttgagcctgaacctcctccagccccaccacgagcttcttctcttggagaaatagatgaatcttctgaattaagagttttggatataccaggacaggattcagagactgtttttgatattcagaatgacagaaatagtatacttccaaaatctcaatctgaatacaagcctgatactcctcagtcaggcctagaatatagtggtattcaagaacttgaggacagaagatctcagcaaaggtttcagtttaatctacaagatttcaggtgttgtgctgtcttgggaagaggacattttggaaaggtgcttttagctgaatataaaaacacaaatgagatgtttgctataaaagccttaaagaaaggagatattgtggctcgagatgaagtagacagcctgatgtgtgaaaaaagaatttttgaaactgtgaatagtgtaaggcatccctttttggtgaacctttttgcatgtttccaaaccaaagagcatgtttgctttgtaatggaatatgctgccggtggggacctaatgatgcacattcatactgatgtcttttctgaaccaagagctgtattttatgctgcttgtgtagttcttgggttgcagtatttacatgaacacaaaattgtttatagagatttgaaattggataacttattgctagatacagagggctttgtgaaaattgctgattttggtctttgcaaagaaggaatgggatatggagatagaacaagcacattttgtggcactcctgaatttcttgccccagaagtattaacagaaacttcttatacaagggctgtagattggtggggccttggcgtgcttatatatgaaatgcttgttggtgagtctccctttcctggtgatgatgaagaggaagtttttgacagtattgtaaatgatgaagtaaggtatccaaggttcttatctacagaagccatttctataatgagaaggctgttaagaagaaatcctgaacggcgccttggggctagcgagaaagatgcagaggatgtaaaaaagcacccatttttccggctaattgattggagcgctctgatggacaaaaaagtaaagccaccatttatacctaccataagaggacgagaagatgttagtaattttgatgatgaatttacctcagaagcacctattctgactccacctcgagaaccaaggatactttcggaagaggagcaggaaatgttcagagattttgactacattgctgattggtgttaagttgctagacactgcgaaaccaagctgactcacaagaagacctcttaaaaatagcaacccttcatttgctctctgtgccaccaatagcttctgagttttttgttgttgttgtttttattgaaacacgtgaagatttgtttaaaagtaccattctaatacttcttcaaaagtggctcctcattgtacttcagcgtaaatatgagcactggaaacagtttcatggagtttaagttgagtgaacatcggccatgaaaatccatcacgaatacttttggatcaatagtctatttt

SEQ ID NO: 10 - protein, 984 aa, Homo sapiens
MASNPERGEILLTELQGDSRSLPFSENVSAVQKLDFSDTMVQQKLDDIKDRIKREIRKELKIKEGAENLRKVTTDKKSLAYVDNILKKSNKKLEELHHKLQELNAHIVVSDPEDITDCPRTPDTPNNDPRCSTSNNRLKALQKQLDIELKVKQGAENMIQMYSNGSSKDRKLHGTAQQLLQDSKTKIEVIRMQILQAVQTNELAFDNAKPVISPLELRMEELRHHFRIEFAVAEGAKNVMKLLGSGKVTDRKALSEAQARFNESSQKLDLLKYSLEQRLNEVPKNHPKSRIIIEELSLVAASPTLSPRQSMISTQNQYSTLSKPAALTGTLEVRLMGCQDILENVPGRSKATSVALPGWSPSETRSSFMSRTSKSKSGSSRNLLKTDDLSNDVCAVLKLDNTVVGQTSWKPISNQSWDQKFTLELDRSRELEISVYWRDWRSLCAVKFLRLEDFLDNQRHGMCLYLEPQGTLFAEVTFFNPVIERRPKLQRQKKIFSKQQGKTFLRAPQMNINIATWGRLVRRAIPTVNHSGTFSPQAPVPTTVPVVDVRIPQLAPPASDSTVTKLDFDLEPEPPPAPPRASSLGEIDESSELRVLDIPGQDSETVFDIQNDRNSILPKSQSEYKPDTPQSGLEYSGIQELEDRRSQQRFQFNLQDFRCCAVLGRGHFGKVLLAEYKNTNEMFAIKALKKGDIVARDEVDSLMCEKRIFETVNSVRHPFLVNLFACFQTKEHVCFVMEYAAGGDLMMHIHTDVFSEPRAVFYAACVVLGLQYLHEHKIVYRDLKLDNLLLDTEGFVKIADFGLCKEGMGYGDRTSTFCGTPEFLAPEVLTETSYTRAVDWWGLGVLIYEMLVGESPFPGDDEEEVFDSIVNDEVRYPRFLSTEAISIMRRLLRRNPERRLGASEKDAEDVKKHPFFRLIDWSALMDKKVKPPFIPTIRGREDVSNFDDEFTSEAPILTPPREPRILSEEEQEMFRDFDYIADWC

SEQ ID NO: 11 - DNA, 839 bp, Homo sapiens
atgtcgggccccaggcctgtggtgctgagcgggccttcgggagctgggaagagcaccctgctgaagaggctgctccaggagcacagcggcatctttggcttcagcgtgtcccataccacgaggaacccgaggcccggcgaggagaacggcaaagattactactttgtaaccagggaggtgatgcagcgtgacatagcagccggcgacttcatcgagcatgccgagttctcggggaacctgtatggcacgagcaaggtggcggtgcaggccgtgcaggccatgaaccgcatctgtgtgctggacgtggacctgcagggtgtgcggaacatcaaggccaccgatctgcggcccatctacatctctgtgcagccgccttcactgcacgtgctggagcagcggctgcggcagcgcaacactgaaaccgaggagagcctggtgaagcggctggctgctgcccaggccgacatggagagcagcaaggagcccggcctgtttgatgtggtcatcattaacgacagcctggaccaggcctacgcagagctgaaggaggcgctctctgaggaaatcaagaaagctcaaaggaccggcgcctgaggcttgctgtctgttctcggcaccccgggcccatacaggaccagggcagcagcattgagccacccccttggcaggcgatacggcagctctgtgcccttggccagcatgtggagtggaggagatgctgcccctgtggttggaacatcctgggtgacccccgacccagcctcgctgggctgtcccctgtccctatctctcactctggacccagggctgacatcctaataaaataactgttggattag

SEQ ID NO: 12 - protein, 197 aa, Homo sapiens
MSGPRPVVLSGPSGAGKSTLLKRLLQEHSGIFGFSVSHTTRNPRPGEENGKDYYFVTREVMQRDIAAGDFIEHAEFSGNLYGTSKVAVQAVQAMNRICVLDVDLQGVRNIKATDLRPIYISVQPPSLHVLEQRLRQRNTETEESLVKRLAAAQADMESSKEPGLFDVVIINDSLDQAYAELKEALSEEIKKAQRTGA

(19) United States
(12) Patent Application Publication
(10) Pub. No.: US 2002/0160438 A1
(43) Pub. Date: Oct. 31, 2002

(54) Method for identifying compounds which positively influence inflammatory conditions

(76) Inventors: Birgit Jung, Schwabenheim (DE); Stefan Mueller, Munchen (DE); Norbert Kraut, Wien (AT)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/029,905

(22) Filed: Dec. 21, 2001

Related U.S. Application Data
(60) Provisional application No. 60/257,854, filed on Dec. 22, 2000.

(51) Int. Cl. C12Q 1/48; C12N 9/12

(52) U.S. Cl. 435/15; 435/194

(57) ABSTRACT

The present invention relates to kinases involved in inflammatory processes and the modulation of the function of such kinases in order to positively influence inflammatory diseases.

### RELATED APPLICATION

The benefit of prior U.S. provisional application no. 60/257,854, filed Dec. 22, 2000 is hereby claimed.

### BACKGROUND

The present invention belongs to the field of modulation of inflammatory processes, in particular of chronic inflammatory airway diseases, in which macrophages play an important role. The inflammatory processes can be modulated according to the invention by influencing the biological activity of a kinase identified to be involved in the inflammatory process.

An example of chronic inflammatory airway disease, in which macrophages play an important role, is chronic bronchitis (CB). CB may occur with or without airflow limitation and includes chronic obstructive pulmonary disease (COPD). CB is a complex disease encompassing symptoms of several disorders: chronic bronchitis which is characterized by cough and mucus hypersecretion, small airway disease, including inflammation and peribronchial fibrosis, emphysema, and airflow limitation. CB is characterized by an accelerated and irreversible decline of lung function. The major risk factor for developing CB is continuous cigarette smoking. Since only about 20% of all smokers are inflicted with CB, a genetic predisposition is also likely to contribute to the disease.

The initial events in the early onset of CB are inflammatory, affecting small and large airways. An irritation caused by cigarette smoking attracts macrophages and neutrophils the number of which is increased in the sputum of smokers. Perpetual smoking leads to an ongoing inflammatory response in the lung by releasing mediators from macrophages, neutrophils and epithelial cells that recruit inflammatory cells to sites of the injury. So far there is no therapy available to reverse the course of CB. Smoking cessation may reduce the decline of lung function.

Only a few drugs are known to date to provide some relief for patients. Long-lasting β2-agonists and anticholinergics are applied to achieve a transient bronchodilation. A variety of antagonists for inflammatory events are under investigation, for example, LTB₄-inhibitors.

There is a continuous need to provide drugs for treating chronic inflammatory airway diseases. Chronic inflammatory airway diseases can be attributed to activated inflammatory immune cells, e.g. macrophages. There is therefore a need for drugs modulating the function of macrophages in order to eliminate a source of inflammatory processes.

### SUMMARY OF THE INVENTION

The present invention relates to methods for determining whether a substance is an activator or an inhibitor of a function of a protein comprising: (a) contacting the protein with a substance to be tested, wherein the protein is a DHAM-kinase; and (b) and measuring whether the function is inhibited or activated, as well as mutants, variants, and fragments of a DHAM-kinase. Such functions may be measured directly or indirectly, and may be made using a cellular or cell-free system. The methods further encompass using mammalian or human DHAM-kinase. The DHAM-kinase may consist of an amino acid sequence of SEQ ID NOs:4, 10, and/or 12, as well as mutants, variants, and fragments thereof. The functions measured by the methods of the invention include kinase activity and substrate binding as well as specific phosphorylation of a substrate.

The present invention also relates to methods for determining an expression level of a DHAM-kinase comprising: (a) determining the level of the DHAM-kinase expressed in a hyperactivated macrophage; (b) determining the level of the DHAM-kinase expressed in a non-hyperactivated macrophage; and (c) comparing the level of the DHAM-kinase expressed in step (a) to the level of the DHAM-kinase expressed in step (b), wherein a difference in levels indicates a differentially expressed DHAM-kinase, as well as mutants, variants, and fragments of a DHAM-kinase, in particular, an amino acid sequence of SEQ ID NOs:4, 10, and/or 12, as well as mutants, variants, and fragments thereof. The level may be determined on a protein or nucleic acid level.

The present invention also relates to methods for diagnosing or monitoring a chronic inflammatory airway disease comprising: (a) determining the level of a DHAM-kinase expressed in a hyperactivated macrophage; (b) determining the level of the DHAM-kinase expressed in a non-hyperactivated macrophage; and (c) comparing the level of the DHAM-kinase expressed in step (a) to the level of the DHAM-kinase expressed in step (b), wherein a difference in levels indicates a differentially expressed DHAM-kinase. The level may be determined on a protein or nucleic acid level.

The present invention also relates to methods for treating a chronic inflammatory airway disease comprising: administering to a subject in need of such treatment an effective amount of a pharmaceutical composition comprising at least one substance determined to be an activator or an inhibitor of a DHAM-kinase.

The present invention also relates to methods for selectively modulating a DHAM-kinase in a macrophage, comprising administering a substance determined to be an activator or an inhibitor of a DHAM-kinase.

The present invention also relates to substances determined to be an activator or an inhibitor of a DHAM-kinase, and pharmaceutical compositions thereof.

The methods and compositions of the invention further relate to chronic inflammatory diseases including, but not limited to, chronic bronchitis and COPD.

### DETAILED DESCRIPTION OF THE INVENTION

In the present invention it was found that macrophages involved in an inflammatory process, particularly in a chronic inflammatory airway disease, more particularly in chronic bronchitis or COPD, show a pattern of differentially expressed nucleic acid sequence and protein expression which differs from the pattern of gene expression of macrophages from healthy donors or donors in an irritated state, which latter do contain macrophages in an activated state. Therefore, macrophages show different activation levels under different inflammatory conditions. For example, it is shown in the present invention that macrophages involved in an inflammatory process in COPD smokers show different gene expression pattern than macrophages from healthy smokers, indicating that in COPD smokers macrophages are in a different, hereinafter named “hyperactivated” or “hyperactive” state. The present invention provides for the inhibition of the hyperactivation or the reduction of the hyperactive state of a macrophage by the identification of substances which modulate kinases involved in the hyperactivation or maintaining the hyperactive state.

The term “chronic inflammatory airway disease” as used hereinafter includes but is not limited to, Chronic Bronchitis (CB) and Chronic Obstructive Pulmonary Disease (COPD). The preferred meaning hereinafter of the term “chronic inflammatory airway disease” is CB and COPD, the more preferred meaning is CB or COPD.

The term “a” as used herein refers to one or more, e.g., “a” DHAM-kinase refers to one or more DHAM-kinases.

The invention is based on the identification of a nucleic acid sequence differentially expressed in a hyperactivated macrophage compared to a macrophage which is not hyperactivated. Such a nucleic acid sequence encodes for a kinase which is involved in the hyperactivation or maintaining the hyperactive state of a macrophage involved in an inflammatory process, preferably in a chronic inflammatory airway disease. Such differentially expressed nucleic acid sequence or protein encoded by such nucleic acid sequence is in the following also named differentially expressed nucleic acid sequence or protein of the invention, respectively. In particular, the present invention teaches a link between phenotypic changes in macrophages due to differentially expressed nucleic acid sequence and protein expression pattern and involvement of macrophages in inflammatory processes and, thus, provides a basis for a variety of applications. For example, the present invention provides a method and a test system for determining the expression level of a macrophage protein of the invention or differentially expressed nucleic acid sequence of the invention and thereby provides e.g. for methods for diagnosis or monitoring of inflammatory processes with involvement of hyperactivated macrophages in mammalian, preferably human beings, especially such beings suffering from an inflammatory process, preferably in a chronic inflammatory airway disease, more preferably in chronic bronchitis or COPD. The invention also relates to a method for identifying a substance by means of a differentially expressed nucleic acid sequence or protein of the invention, which substance modulates, i.e. acts as an inhibitor or activator of the said differentially expressed nucleic acid sequence or protein of the invention and thereby positively influences chronic inflammatory processes by inhibition of the hyperactivation or reduction of the hyperactive state of macrophages, and thereby allows treatment of mammals, preferably human beings, suffering from a said disease. The invention also relates to a method for selectively modulating such a differentially expressed nucleic acid sequence or protein of the invention in a macrophage comprising administering a substance determined to be a modulator of said protein or differentially expressed nucleic acid sequence. The present invention includes the use of said substances for treating beings in need of a treatment for an inflammatory process.

In the present invention in a first step a differentially expressed nucleic acid sequence of the invention is identified which has a different expression pattern in a hyperactivated macrophage compared to a macrophage which is not hyperactivated. For the sake of conciseness this description deals particularly with investigation of macrophages involved in COPD, however, equivalent results may be obtained with samples from subjects suffering from other chronic inflammatory airway diseases, e.g. other chronic bronchitis symptoms. The investigation of the different expression pattern leads to the identification of a series of differentially expressed nucleic acid sequences expressed in dependency on the activation state of a macrophage involved in an inflammatory process, as exemplified in the Examples hereinbelow.

Briefly, such a differentially expressed nucleic acid sequence of the invention is identified by comparative expression profiling experiments using a cell or cellular extract from a hyperactivated macrophage, i.e. for example from the site of inflammation in COPD and from the corresponding site of control being not suffering from said disease, however, suffering under the same irritating condition, for example, cigarette smoke exposure.

In a second step the proteins are identified which are encoded by the differentially expressed nucleic acid sequence, i.e. proteins playing a role in mediating the hyperactivation or in maintaining the hyperactivated state. A class of differentially expressed nucleic acid sequences of the invention can be identified to encode a class of kinases which is characterized in that it is expressed in a macrophage that is hyperactivated according to the invention at a lower or higher level than the control level in a macrophage which is not hyperactivated. Such a kinase of the invention is hereinafter named DHAM-kinase (“deregulated in hyperactive macrophage”-kinase).

A preferred example of a DHAM kinase according to the present invention is Guanylate kinase 1 (GUK1)(Brady, W. A. et al. (1996) J. Biol. Chem. 271, 16734-16740); Serine-Threonine-Kinase PAK2 (Knaus, U. G. et al. (1995) Science 269, 221-223; Frost, J. A. et al. (1996) Mol. Cell. Biol. 16, 3707-3713; Goeckeler, Z. M. et al. (2000) J. Biol. Chem. 275, 18366-18374; Zeng, Q. et al. (2000) J. Cell Sci. 113, 471-482), or Serine-Threonine-Kinase PRK2 (Vincent, S. and J. Settleman (1997) Mol. Cell. Biol. 17, 2247-2256), depicted in the sequence listing.

The biological activity of a DHAM-kinase according to the present invention, i.e. mediating the involvement of a macrophage in an inflammatory process according to the invention, is dependent, for example, on substrate phosphorylation and/or on other DHAM-kinase functions such as substrate recognition and/or substrate binding.

The invention also concerns functional equivalents, derivatives, variants, mutants and fragments of a DHAM-kinase, preferentially of the preferred kinases mentioned hereinbefore. Functional in this context means having a function of the respective corresponding DHAM-kinase which is involved in its biological activity, e.g. substrate phosphorylation, recognition, and /or binding.

According to the present invention, the biological activity of a DHAM-kinase expressed at a lower level than the control level is preferably activated in order to inhibit hyperactivation or reduce a hyperactivated state of a macrophage; the biological activity of a DHAM-kinase which is expressed at a higher level than the control level is preferably inhibited in order to inhibit hyperactivation or reduce a hyperactivated state of a macrophage.

In one embodiment the present invention concerns a test method for determining whether a substance is an activator or inhibitor of a DHAM-kinase. Since a DHAM-kinase is involved in chronic inflammatory airway disease and plays a role in mediating inflammation, a substance modulating the biological activity of a DHAM-Kinase can be used for treating a chronic inflammatory airway disease or can be used as a lead compound for optimization of the function of the substance in a way that the optimized substance is suitable for treating chronic inflammatory airway diseases. For performing a method of the invention, a test system according to the invention can be used.

The present invention also concerns a test system for determining whether a substance is an activator or an inhibitor of a DHAM-kinase. A test system useful for performing a method of the invention comprises a cellular or a cell-free system. For example, one embodiment of the invention concerns a test system that is designed in a way to allow the testing of substances acting on the expression level of the differentially expressed nucleic acid sequence e.g. using expression of a reporter-gene, e.g. luciferase gene or the like, as a measurable readout. Another embodiment of the invention concerns a test system that is designed in a way to allow the testing of substances directly interacting with a function, e.g. the enzymatic activity, of the DHAM-kinase or interfering with the activation of a function, e.g. enzymatic activity, of the DHAM-kinase by a natural or an artificial but appropriate activator of the DHAM-Kinase, e.g. an appropriate kinase or the like.

A test system according to the invention comprises a DHAM-kinase, or a functional equivalent, derivative, variant, mutant or fragment of a DHAM-kinase, a nucleic acid encoding a said protein or encoding a functional equivalent, derivative, variant, mutant or fragment of a DHAM-kinase and/or regulatory elements, wherein a functional equivalent, derivative, variant, mutant or fragment of a DHAM-kinase or a nucleic acid encoding a DHAM-kinase or a functional equivalent, derivative, variant, mutant or fragment of a DHAM-kinase is able to interact with a substance which can be tested in a way that direct interaction leads to a measurable read-out indicative for the change of a respective biological activity of a DHAM-kinase and/or for the change of expression of a DHAM-kinase.

A test system of the invention comprises, for example, elements well known in the art. Cell-free systems may include, for example, a DHAM-kinase or a functional equivalent, derivative, variant, mutant or fragment of a DHAM-kinase, a nucleic acid encoding a DHAM-kinase or encoding a functional equivalent, derivative, variant, mutant or fragment of a DHAM-kinase in soluble or bound form or in cellular compartments or vesicles. Suitable cellular systems include, for example, a suitable prokaryotic cell or eukaryotic cell, e.g. comprising a DHAM-kinase or a functional equivalent, derivative, variant, mutant or fragment of a DHAM-kinase, a nucleic acid encoding a DHAM-kinase or encoding a functional equivalent, derivative, variant, mutant or fragment of DHAM-kinase (Tsuchiya, S. et al. (1980) Int.J. Cancer 26, 171-176; Ziegler-Heitbrock, H. W. et al. (1988) lnt.J.Cancer 41, 456-461). A cell suitable for use in a said test system of the invention may be obtained by recombinant techniques, e.g. after transformation or transfection with a recombinant vector suitable for expression of a desired DHAM-kinase or functional equivalent, derivative, variant, mutant or fragment of a DHAM-kinase, or may be e.g. a cell line or a cell isolated from a natural source expressing a desired DHAM-kinase or functional equivalent, derivative, variant, mutant or fragment of DHAM-kinase. A test system of the invention may include a natural or artificial ligand of a DHAM-kinase if desirable or necessary for testing whether a substance of interest is an inhibitor or activator of a DHAM-kinase.

A test method according to the invention comprises measuring a read-out, e.g. a phenotypic change in the test system, for example, if a cellular system is used a phenotypic change of the cell is monitored. Such change may be a change in a naturally occurring or artificial response of the cell to DHAM-kinase activation or inhibition, e.g. as detailed in the Examples hereinbelow.

A test method according to the invention can on the one hand be useful for high throughput testing suitable for determining whether a substance is an inhibitor or activator of the invention, but also e.g. for secondary testing or validation of a hit or lead substance identified in high throughput testing.

The present invention also concerns a substance identified using a method according to the invention to be an inhibitor or activator of a DHAM-kinase of the invention. A substance of the present invention is any compound which is capable of activating or preferably inhibiting a function of a DHAM-kinase according the invention. An example of a way to activate or inhibit a function of a DHAM-kinase is by influencing the expression level of said DHAM-kinase. Another example of a way to activate or inhibit a function of a DHAM-kinase is to apply a substance which directly binds the DHAM-kinase and thereby activates or blocks functional domains of said DHAM-kinase, which can be done reversibly or irreversibly, depending on the nature of the substance applied.

Accordingly, a substance useful for activating or inhibiting biological activity of a DHAM-kinase includes a substance acting on the expression of a differentially expressed nucleic acid sequence, for example a nucleic acid fragment hybridizing with the corresponding gene or regulatory sequence and thereby influencing gene expression, or a substance acting on a DHAM-kinase itself or on its activation or inhibition by other naturally occurring cellular components, e.g. another protein acting enzymatically on a said protein of the invention, e.g. a protein kinase.

Therefore, the invention concerns, for example, a substance which is a nucleic acid sequence coding for a DHAM-kinase, or a fragment, derivative, mutant or variant of such a nucleic acid sequence, which nucleic acid sequence or a fragment, derivative, mutant or variant thereof is capable of influencing the gene expression level, e.g. a nucleic acid molecule suitable as antisense nucleic acid, ribozyme, or for triple helix formation.

The invention also concerns a substance which is e.g. an antibody or an organic or inorganic compound which directly binds to or interfers with the activation of a DHAM-kinase and thereby affects its biological activity.

In a further aspect, the present invention relates to a method for determining an expression level of a nucleic acid coding for a DHAM-kinase, preferably messenger RNA, or protein of the invention itself, in a cell, preferably in a macrophage, more preferably in a macrophage isolated from a site of inflammation, even more preferably from a site of inflammation in a subject suffering from a chronic inflammatory airway disease. Such a method can be used, for example, for testing whether a substance is capable of influencing differentially expressed nucleic acid sequence expression levels in a method outlined above for determining whether a substance is an activator or inhibitor according to the present invention. A method for determining an expression level according to the invention can, however, also be used for testing the activation state of a macrophage, e.g. for diagnostic purposes or for investigation of the success of treatment for a disease which is caused by the hyperactivated macrophage. Said macrophage is preferably a mammalian, more preferably a human cell. Accordingly, macrophages of the present invention are preferably obtainable from the site of inflammation in a mammal and more preferably from a site of inflammation in a human being.

Accordingly, the invention also relates to a method for diagnosis of a chronic inflammatory disease, or monitoring of such disease, e.g. monitoring success in treating beings in need of treatment for such disease, comprising determining an expression level of a nucleic acid coding for a DHAM-kinase, preferably messenger RNA, or a DHAM-kinase itself in a macrophage.

A method for determining expression levels of a nucleic acid coding for a protein of the invention, preferably messenger RNA, or protein of the invention itself can, depending on the purpose of determining the expression level, be performed by known procedures such as measuring the concentration of respective RNA transcripts via hybridization techniques or via reporter gene driven assays such as luciferase assays or by measuring the protein concentration of said protein of the invention using respective antibodies.

The present invention also relates to the use of a substance according to the invention for the treatment of a chronic inflammatory airway disease. Another embodiment of the present invention relates to a pharmaceutical composition comprising at least one of the substances according to the invention determined to be an activator or an inhibitor. The composition may be manufactured in a manner that is itself known, e.g. by means of conventional mixing, dissolving, granulating, dragee-making, levigating, powdering, emulsifying, encapsulating, entrapping or lyophilizing processes.

In order to use substances which activate or inhibit according to the invention as drugs for treatment of chronic inflammatory airway diseases, the substances can be tested in animal models, for example, an animal suffering from an inflammatory airway disorder or a transgenic animal expressing a DHAM-kinase according to the invention.

Toxicity and therapeutic efficacy of a substance according to the invention can be determined by standard pharmaceutical procedures, which include conducting cell culture and animal experiments to determine the IC₅₀, LD₅₀ and ED₅₀. The data obtained are used for estimating the animal or more preferred the human dose range, which will also depend on the dosage form (tablets, capsules, aerosol sprays ampules, etc.) and the administration route (for example transdermal, oral, buccal, nasal, enteral, parenteral, inhalative, intratracheal, or rectal).

A pharmaceutical composition containing at least one substance according to the invention as an active ingredient can be formulated in conventional manner. Methods for making such formulations can be found in manuals, e.g. “Remington Pharmaceutical Science”. Examples for ingredients that are useful for formulating at least one substance according to the present invention are also found in WO 99/18193, which is hereby incorporated by reference.

In a further aspect the invention concerns a method for treating a chronic inflammatory airway disease according to the invention. Such method comprises administering to a being, preferably to a human being, in need of such treatment a suitable amount of a pharmaceutical composition comprising at least one substance determined to be an activator or inhibitor by a method according to the invention for determining whether a substance is an activator or an inhibitor of a DHAM-kinase.

In another embodiment the invention relates to a method for selectively modulating DHAM-kinase concentration in a macrophage, comprising administering a substance determined to be an activator or inhibitor of a DHAM-kinase according to the invention.

Included herein are exemplified embodiments, which are intended as illustrations of single aspects of the invention. Indeed, various modifications of the invention in addition to those herein will become apparent to those skilled in the art from the foregoing description. Such modifications are intended to fall within the scope of the present invention.

All publications and patent applications cited herein are incorporated by reference in their entireties.

### EXAMPLES

### Example 1

The following is an illustration of how comparative expression profiling can be performed in order to identify a DHAM-kinase according to the present invention.

1.1. Selection of Subjects

Three groups of subjects are studied: healthy non-smokers, healthy smokers and patients with COPD.

In order to assess lung function subjects have to perform spirometry. A simple calculation based on age and height is used to characterize the results. COPD subjects are included if their FEV₁ % (forced expriatory volume, 1 second) predicted is less than 70%. Healthy smokers are age and smoking history matched with the COPD subjects but have normal lung function. Healthy non-smokers have normal lung function and have never smoked. The latter group has a methacholine challenge to exclude asthma. This technique requires increasing doses of methacholine to be given to the subject, with spirometry between each dose. When the FEV, falls 20% the test is stopped and the PC₂₀ is calculated. This is the dose of methacholine causing a 20% fall in FEV₁ and we require a value of greater than 32 as evidence of absence of asthma. All subjects have skin prick tests to common allergens and are required to have negative results. This excludes atopic individuals. The clinical history of the subjects is monitored and examined in order to exclude concomitant disease.

1.2. BAL (Bronchoalveolar Lavage) Procedure

Subjects are sedated with midazolam prior to the BAL. Local anesthetic spray is used to anaesthetize the back of the throat. A 7mm Olympus bronchoscope is used. The lavaged area is the right middle lobe. 250 ml of sterile saline is instilled and immediately aspirated. The resulting aspirate contains macrophages.

1.3. BAL Processing

BAL is filtered through sterile gauze to remove debris. The cells are washed twice in HBSS (Hank's Balanced Salt Solution), resuspended in 1 ml HBSS and counted. The macrophages are spun to a pellet using 15 ml Falcon blue-cap polypropylene, resuspended in Trizol reagent (Gibco BRL Life Technologies) at a concentration of 1 ml Trizol reagent per 10 million cells and then frozen at −70° C.

1.4. Differential Gene Expression Analysis

Total RNA is extracted from macrophage samples obtained according to Example 1.3. Cell suspensions in Trizol are homogenized through pipetting and incubated at room temperature (RT) for 5 minutes. 200 μl chloroform per ml Trizol is added, the mixture carefully mixed for 15 seconds and incubated for 3 more minutes at room temperature. The samples are spun at 10,000 g for 15 minutes at 4° C. The upper phase is transferred into a new reaction tube and the RNA is precipitated by adding 0.5 ml isopropanol per ml Trizol for 10 minutes at room temperature. Then, the precipitate is pelleted by using a microcentrifuge for 10 minutes at 4° C. with 10,000 g; the pellet is washed twice with 75% ethanol, air dried and resuspended in DEPC-H₂O.

An RNA cleanup with Qiagen RNeasy Total RNA isolation kit (Qiagen) is performed in order to improve the purity of the RNA. The purity of the RNA is determined by agarose gel electrophoresis and the concentration is measured by UV absorption at 260 nm.

5 μg of each RNA is used for cDNA synthesis. First and second strand syntheses are performed with the SuperScript Choice system (Gibco BRL Life Technologies). In a total volume of 11 μl RNA and 1 μM of 100 μM T7−(dt)₂₄ primer, sequence shown in SEQ ID NO:1, RNA and primer are heated up to 70° C. for 10 minutes and then cooled down on ice for 2 minutes. First strand buffer to a final concentration of 1×, DTT to a concentration of 10 mM and a dNTP mix to a final concentration of 0.5 mM are added to a total volume of 18 μl. The reaction mix is incubated at 42° C. for 2 minutes and 2 μl of Superscript II reverse transcriptase (200 U/μl) are added. For second strand synthesis, 130 μl of a mix containing 1.15× second strand buffer, 230 μM dNTPs, 10 U E. coil DNA ligase (10U/μl), E. coil DNA polymerase (10 U/μl), RNase H (2U/μl) are added to the reaction of the first strand synthesis and carefully mixed with a pipette. Second strand synthesis is performed at 16° C. for 2 hours, then 2 μl of T4 DNA polymerase (5 U/μl) are added, incubated for 5 minutes at 16° C. and the reaction is stopped by adding 10 μl 0.5 M EDTA.

Prior to cRNA synthesis, the double stranded cDNA is purified. The cDNA is mixed with an equal volume of phenol:chloroform:isoamylalcohol (25:24:1) and spun through the gel matrix of phase lock gels (Eppendorf) in a microcentrifuge in order to separate the cDNA from unbound nucleotides. The aqueous phase is precipitated with ammonium acetate and ethanol. Subsequently, the cDNA is used for in vitro transcription. cRNA synthesis is performed with the ENZO BioArray High Yield RNA Transcript Labeling Kit according to manufacturer's protocol (ENZO Diagnostics). Briefly, the cDNA is incubated with 1× HY reaction buffer, 1× biotin labeled ribonucleotides, 1× DTT, 1× RNase Inhibitor Mix and 1× T7 RNA Polymerase in a total volume of 40 μl for 5 hours at 37° C. Then, the reaction mix is purified via RNeasy columns (Qiagen), the cRNA is precipitated with ammonium acetate and ethanol and finally resuspended in DEPC-treated water. The concentration is determined via UV spectrometry at 260 nm. The remaining cRNA is incubated with 1× fragmentation buffer (5× fragmentation buffer: 200 mM Tris acetate, pH 8.1, 500 mM KOAc, 150 mM MgOAc) at 94° C. for 35 minutes.

For hybridization of the DNA chip, 15 μg of cRNA is used, mixed with 50 pM biotin-labeled control B2 oligonucleotide, sequence shown in SEQ ID NO:2, 1× cRNA cocktail, 0.1 mg/ml herring sperm DNA, 0.5 mg/ml acetylated BSA, 1× MES (2-[N-morpholino]-ethanesulfonic acid) hybridization buffer in a total volume of 300 μl. The hybridization mixture is heated up to 99° C. for 5 minutes, cooled down to 45° C. for 10 minutes and 200 μl of the mix are used to fill the probe array. The hybridization is performed at 45° C. at 60 rpm for 16 hours.

After the hybridization the hybridization mix on the chip is replaced by 300 μl on-stringent wash buffer (100 mM MES, 100 mM NaCl, 0.01% Tween 20). The chip is inserted into an Affymetrix Fluidics station and washing and staining is performed according to the EukGE-WS2 protocol. The staining solution per chip consists of 600 μl 1× stain buffer (100 mM MES, 1 M NaCl, 0.05% Tween 20), 2 mg/ml BSA, 10 μg/ml SAPE (streptavidin phycoerythrin) (Dianova); the antibody solution consists of 1× stain buffer, 2 mg/ml BSA, 0.1 mg/ml goat IgG, 3 μg/ml biotinylated antibody. After the washing and staining procedure, the chips are scanned on the HP Gene Array Scanner (Hewlett Packard).

Data Analysis is performed by pairwise comparisons between chips hybridized with RNA isolated from COPD smokers and chips hybridized with RNA isolated from healthy smokers.

The following is an illustration of differentially expressed genes and their function as identified according to the approach of the present invention.

### Example 2

PAK2

A gene that is identified as consistently downregulated in individuals with COPD codes for PAK2 (SEQ ID NOs:3, 4). PAK2 is a serine/threonine kinase that preferentially interacts with activated Cdc42 and Rac, but not Rho. This association leads to autophosphorylation of PAK2 and activation of its kinase activity. PAK2 can phosphorylate myosin II, MLCK (myosin light chain kinase), p47phox (NADPH oxidase), and Raf-1. PAK2 is involved in actin reorganization and cell motility (Knaus, U. G. et al. (1995) Science 269, 221-223; Frost, J. A. et al. (1996) Mol. Cell. Biol. 16, 3707-3713; Goeckeler, Z. M. et al. (2000) J. Biol. Chem. 275,18366-18374; Zeng, Q. et al. (2000) J. Cell Sci. 113, 471-482).

PAK2 is consistently found downregulated (47%) in COPD smokers compared to healthy smokers. This is shown by “avg diff” values (Table 1) and “fold change” values (Table 2 ). The p values for two separate groups comparing COPD smokers and healthy smokers are 0.001 and 0.004.

Table 1A and 1B: Expression levels of PAK2: “avg diff” values for each patient are listed as well as mean and median values for the three groups of subjects; OS means obstructed smoker, HS healthy smoker, NS non-smoker

TABLE 1A

| Subject | | Subject | |
|---|---|---|---|
| (OS) | Avg diff | (HS) | Avg Diff |
| P01 | 912.4 | P02 | 752.3 |
| P03 | 813.5 | P37 | 965.7 |
| P05 | 427.3 | P43 | 1284.0 |
| P06 | 511.0 | P56 | 1180.5 |
| P39 | 443.5 | P57 | 1143.0 |
| P44 | 519.8 | P58 | 1215.2 |
| | | P62 | 1586.4 |
| Mean ± | 604.6 ± 205.8 | | 1161.0 ± 259.8 |
| SD | | | |
| Median | 515.4 | | 1180.5 |

TABLE 1B

| Subject | | Subject | |
|---|---|---|---|
| (OS) | Avg diff | (HS) | Avg Diff |
| P64 | 570.2 | P65 | 798.4 |
| P68 | 403.2 | P66 | 1282.1 |
| P70 | 612.2 | P69 | 1066.2 |
| P71 | 404.9 | P76 | 771.7 |
| | | P78 | 951.0 |
| Mean ± | 497.6 ± 54.7 | | 973.9 ± 93.7 |
| SD | | | |
| Median | 487.6 | | 798.4 |

Table 2: Fold change values (FC) for comparisons between obstructed smoker and healthy smokers. On average PAK2 is downregulated by 1.78 fold, the median is 1.9 fold.

TABLE 2

| comp | FC | comp | FC | comp | FC | Comp | FC |
|---|---|---|---|---|---|---|---|
| 1 vs 2 | 1.2 | 5 vs 43 | −2.5 | 39 vs 57 | −2.2 | 68 vs 66 | −2.8 |
| 1 vs 37 | −1.1 | 5 vs 56 | −2.0 | 39 vs 58 | −2.5 | 68 vs 69 | −2.3 |
| 1 vs 43 | −1.2 | 5 vs 57 | −2.1 | 39 vs 62 | −2.9 | 68 vs 76 | −1.9 |
| 1 vs 56 | −1.1 | 5 vs 58 | −2.3 | 44 vs 2 | −1.7 | 68 vs 78 | −2.0 |
| 1 vs 57 | −1.1 | 5 vs 62 | −2.8 | 44 vs 37 | −1.9 | 70 vs 65 | −1.3 |
| 1 vs 58 | −1.2 | 6 vs 2 | −1.5 | 44 vs 43 | −2.1 | 70 vs 66 | −1.8 |
| 1 vs 62 | −1.4 | 6 vs 37 | −1.9 | 44 vs 56 | −1.9 | 70 vs 69 | −1.5 |
| 3 vs 2 | 1.1 | 6 vs 43 | −2.2 | 44 vs 57 | −1.9 | 70 vs 76 | −1.3 |
| 3 vs 37 | −1.2 | 6 vs 56 | −1.9 | 44 vs 58 | −2.1 | 70 vs 78 | −1.3 |
| 3 vs 43 | −1.4 | 6 vs 57 | −1.9 | 44 vs 62 | −2.5 | 71 vs 65 | −2.2 |
| 3 vs 56 | −1.2 | 6 vs 58 | −2.1 | 64 vs 65 | −1.5 | 71 vs 66 | −2.8 |
| 3 vs 57 | −1.2 | 6 vs 62 | −2.5 | 64 vs 66 | −2.0 | 71 vs 69 | −2.2 |
| 3 vs 58 | −1.3 | 39 vs 2 | −2.0 | 64 vs 69 | −1.6 | 71 vs 76 | −2.0 |
| 3 vs 62 | −1.6 | 39 vs 37 | −2.2 | 64 vs 76 | −1.4 | 71 vs 78 | −2.0 |
| 5 vs 2 | −1.7 | 39 vs 43 | −2.5 | 64 vs 78 | −1.4 | | |
| 5 vs 37 | −2.1 | 39 vs 56 | −2.2 | 68 vs 65 | −2.0 | | |

2.1. Cloning of PAK2

PAK2 is cloned from total RNA extracted from human PMNs (polymorphonuclear neutrophils) isolated from healthy volunteers. 5 μg RNA is reverse transcribed into cDNA with 5 ng oligo (dt)₁₈ primer, 1× first strand buffer, 10 mM DTT, 0.5 mM dNTPs and 2 U Superscript II (Gibco BRL) at 42° C. for 50 minutes. Then, the reaction is terminated at 70° C. for 15 minutes and the cDNA concentration is determined by UV-spectrophotometry. For amplification of PAK2, 100 ng of the cDNA and 10 pmoles of sequence-specific primers for PAK2 (SEQ ID NO:5 forward primer and SEQ ID NO:6 reverse primer) are used for PCR. Reaction conditions are: 2 minutes at 94° C., 35 cycles with 30 seconds at 94° C., 30 seconds at 53° C., 90 seconds at 72° C., followed by 7 minutes at 72° C. with Taq DNA-polymerase. The reaction mix is separated on a 2% agarose gel, a band of about 1,000 bp is cut out and purified with the QIAEX II extraction kit (Qiagen). The concentration of the purified band is determined and about 120 ng are incubated with 300 ng of pDONR201, the donor vector of the Gateway system (Life Technologies), 1× BP clonase reaction buffer, BP clonase enzyme mix in a total volume of 20 μl for 60 minutes at 25° C. Then, reactions are incubated with 2 μl of proteinase K and incubated for 10 minutes at 37° C. The reaction mix is then electroporated into competent DB3.1 cells and plated on Kanamycin-containing plates. Clones are verified by sequencing. A clone, designated pDONR-PAK2, with identical sequence to the database entry (accession no. U24153) is used for further experiments.

2.2 PAK2 Expression Vector

The vector containing PAK2 described above is used to transfer the CDNA for PAK2 to the expression vector pcDNA3.1(+)/attR that contains the “attR1” and “attR2” recombination sites of the Gateway cloning system (Life Technologies) where PAK2 is expressed under the control of the CMV promoter. 150 ng of the “entry vector” pDONR-PAK2 is mixed with 150 ng of the “destination vector” pcDNA3.1(+)/attR, 4 μl of the LR Clonase enzyme mix, 4 μl LR Clonase reaction buffer, added up with TE (Tris/EDTA) to 20 μl and incubated at 25° C. for 60 minutes. Then, 2 μl of proteinase K solution is added and incubated for 10 minutes at 37° C. 1 μl of the reaction mix is transformed into 50 μl DH5α by a heat-shock of 30 seconds at 42° C. after incubating cells with DNA for 30 minutes on ice. After heat-shock of the cells, 450 μl of S.O.C. is added and cells are incubated at 37° C. for 60 minutes. Cells (100 μl) are plated on LB plates containing 100 μg/ml ampicillin and incubated overnight.

A colony that contains pcDNA3.1(+)/attR with PAK2 as an insert is designated pcDNA/PAK2 and used for transfection studies.

A similar cloning reaction is performed with a constitutively active mutant of PAK2. This mutant is generated by replacing the nucleotides ACC (position 1420-1422 of the coding region) with GAA. Thereby, the amino acid threonine at position 461 is replaced by glutamic acid. The clone is called pcDNA/PAK2T461 E.

2.3. Myc-tagged expression vector for PAK2

In order to generate a C-terminal Myc-tagged version of PAK2, the coding sequence of PAK2 devoid of the stop codon is amplified by PCR according to the reaction conditions indicated above with forward primer SEQ ID NO:7 and reverse primer SEQ ID NO:8. The PCR product is digested with EcoRI and Xbal, separated on a 1% agarose gel, cut out and purified with the QIAEX II extraction kit (Qiagen). The product is then cloned in frame into pcDNA3.1/myc-His (Clontech), that is digested with EcoRI and Xbal. Similarly, the coding sequence of the constitutively active mutant of PAK2 is cloned into pcDNA3.1/myc-His.

2.4. Purification of Myc-tagged PAK2

For immunoprecipitation of Myc-tagged PAK2, anti-myc mouse monoclonal antibodies (9E10) (Santa Cruz Biotechnology) are used that are coupled to Dynabeads M-280 (Dynal). Dynabeads are preincubated in buffer A (20 mM Tris/HCl, pH 8, 0.2 mM EDTA, 10% glycerol, 5 mM MgCl₂, 100 mM KCl) in the presence of 1 mg/ml BSA for 10 minutes. Beads are washed twice and resuspended in the same volume as before incubation in buffer A. Coupling is performed for 2 hours at room temperature with 5 μg of anti-myc antibodies and 50 μl of Dynabeads M-280. Then, beads are washed three times with 500 μl RIPA buffer (10 mM Tris/HCl, pH 8,140 mM NaCl, 1 mM EDTA, 1% NP40, 0.1% SDS, 1% deoxycholate), followed by two washes with buffer A. Beads are then incubated for 2 hours at 4° C. with 300 μl of cytosolic extract containing myc-tagged PAK2. Beads are collected with the magnetic device and washed 4 times in ice-cold kinase buffer (50 mM Tris/HCl, pH 7.5, 5 mM MgCl₂, 1 mM EDTA, 1 mM EGTA, 10 mM β-mercaptoethanol, phosphatase inhibitors (50 mM NaF, 5 mM Na₄P₂O₇, 2 mM Na₃VO₄, 10 nM okadaic acid) and protease inhibitors (40 μg/ml leupeptin, 40 μg/ml pepstatin, 40 μg/ml aprotinin, 500 μpM PMSF (phenylmethylsulfonyl fluoride).

2.5. Transfection of THP-1 cells with PAK2-constructs

THP-1 cells are grown in RPMI 1640 media (Bio Whittaker), containing 10% FCS supplemented with 100 U/ml penicillin, 100 μg/ml streptomycin, 2 mM glutamine, and 1× non-essential amino acids in a humidified atmosphere with 5% CO₂ at 37° C. 2−5×10⁵ cells of freshly passaged THP-1 cells are seeded in a 35 mm Petri dish in a culture volume of 2 ml.

6 μl FuGene6 (Roche Biochemicals) is added to 100 μl of culture media without serum and equilibrated for 5 minutes at room temperature. Then, 2 μg of purified pcDNA/PAK2 or pcDNA/PAK2T461E is added to the prediluted FuGene6 solution, gently mixed, and further incubated at room temperature for 15 minutes. Then, the FuGene6/DNA solution is added dropwise to the cells and distributed evenly by swirling of the media. After 24 hours the media is replaced by media containing 200 μg/ml G418.

In order to generate stable clones expressing PAK2 or PAK2T461E, cells are spun down after 48 hours for 5 minutes at room temperature at 500 xg. The media is aspirated and replaced by RPMI 1640, 10% FCS, 2 mM glutamine, 100 U/ml penicillin,100 ,g/ml streptomycin, and 200 μg/ml G418. During the following five days the media is replaced daily until dead cells and debris are washed away. Single colonies are isolated by limited dilution into 394-well plates. Single clones are expanded and the expression of PAK2 in several clones is tested via PAK2-specific antibodies (clone V-19, Santa Cruz Biotechnology).

2.6. Autophosphorylation of PAK2

Autophosphorylation of PAK2 is induced by activated Cdc42. Therefore 500 ng of Cdc42 is preloaded with 180 μM GTPγS (Roche Biochemicals) for 10 minutes at 30° C. For autophosphorylation, 500 ng of myc-taggedPAK2 or PAK2T461E are incubated in a reaction volume of 20 μl in 50 mM Tris/HCl, pH 7.4,10 mM MgCl₂, 30 mM β-mercaptoethanol, 0.2 mM [γ-³²P]ATP (1000 cpm/pmol) (Amersham). 500 ng of GTPγS-loaded Cdc42 and substances according to the invention in a concentration range from 0.5 to 300 nM are added and incubated for 30 minutes at 30° C. The reactions are stopped by adding 10 ml of trichloroacetic acid (30%), filtered through GF/B glass fiber filters (Whatman) on a Packard cell harvester, and washed twice with 50 mM Tris/HCl, pH 7.4,10 mM MgCl₂, 30 mM β-mercaptoethanol. After adding 30 μl of Microscint cocktail (Packard) filter-bound radioactivity is counted in a microplate scintillation counter.

2.7. Phosphorylation of Histone h4

In order to activate PAK2, PAK2 is induced by activated Cdc42. Therefore, 500 ng of Cdc42 is preloaded with 180 μM GTPγS (Roche Biochemicals) for 10 minutes at 30° C. For autophosphorylation, 500 ng of myc-tagged PAK2 or PAK2T461E are incubated in a reaction volume of 20 μl in 50 mM Tris/HCl, pH 7.4,10 mM MgCl₂, 30 mM β-mercaptoethanol, 0.2 mM [β-³²P]ATP (1,000 cpm/pmol) (Amersham). 500 ng of GTPβS-loaded Cdc42, 20 mg histone h4 (Sigma) and substances according to the invention in a concentration range from 0.5 to 300 nM are added and incubated for 30 minutes at 30° C. The reactions are stopped by adding 2× Laemmli buffer and the reaction mixes are separated on 12% SDS polyacrylamide gels (Biorad). Radioactivity incorporated into histone h4 is determined by phosphor imaging (Storm 860, Molecular Dynamics).

2.8. SPA-Assay (Scintillation Proximity Assay) for Kinase-activity

The assay is performed in 384-well plates (Packard Optiplate, white, flat bottom, Prod.-No. 6005214). Histone with a biotin at the N-terminus is used as a substrate for recombinant PAK2. The enzyme stock is stored in a 50 mM Tris/0.1 mM EGTA/0.1% 2-mercaptoethanol/10 mM magnesium acetate/0.1 mM ATP/ pH 7.5 and stored in aliquots at −80° C.

Method:

In the 384-well plates, 10 μl test compound in demineralized water (containing 5% DMSO, final concentration 1%) are mixed with 15 μl PAK2 (1 U/ml μM; f.c. 0.3 U/ml) in enzyme dilution buffer (1 mg/ml BSA/ 50 mM Tris/ 0.1 mM EGTA/0.1% 2-mercaptoethanol/ pH 7.5) and incubated for 15 min at room temperature. For the “negative” controls (100% CTL, non-inhibited enzyme activity), the test compound is omitted from the above mixture. For the “positive” controls (0% CTL, fully inhibited enzyme activity), the test compound is replaced by staurosporine (100 μM, f.c. 20 μM). The biotinylated histone (1.5 μM, f.c. 0.75 μM) and γ-³³P-labelled ATP (0.17 μCi/well) are added in 25 μl of a 2× kinase assay buffer (50 mM Tris/ 10 mM beta-glycerophosphate/ 4 mM dithiothreitol/ 200 μM sodium vanadate/ 20 mM MgCl2/pH7.5). The plates are then incubated at room temperature for 2 hours. After the incubation period, 0.1 mg/well of LEADseeker streptavidin-coated polystyrene beads are added in 30 μl of a solution containing 100 mM Tris/10 mM EDTA/100 pM cold ATP. After 1 h of incubation at RT, the plates are centrifuged for 1 min at 500 g.

Each assay microtiter plate contains wells with “negative” and “positive” controls as described above. The analysis of the data is performed by the calculation of the percentage of scintillation in the presence of the test compound compared to the scintillation of the “negative” control after subtracting the “positive” control:

%CTL=(scintillation (sample)−scintillation (“positive” control))*100/(scintillation (“negative” control)−scintillation (“positive” control)).

An inhibitor of the PAK2 enzyme will give values between 100% CTL (no inhibition) and 0% CTL (complete inhibition). Values of more than 100% CTL are normally related to compound-specific physico-chemical properties or indirect biochemical effects such as allosteric regulation.

2.9. Phenotypic/Cellular Effects Caused by PAK2

The following assays are performed with cell lines THP-1 Tsuchiya, S. et al. (1980) lnt.J. Cancer 26, 171-176) or MonoMac 6 (Ziegler-Heitbrock, H. W. et al. (1988) Int.J.Cancer 41, 456-461) that are transiently or stably transfected with PAK2 or PAK2/T461E and the read-outs are compared to mock-transfected cells. In addition, substances according to the invention that stimulate the activity of PAK2 are added.

Production and Release of Cytokines

Monocytic/macrophage cell lines are stimulated with various stimuli, such as 10 nM PMA, 20 ng/ml M-CSF, 20 ng/ml GM-CSF, 20 μg/ml LPS (from Salmonella minnesota Re595) at cell densities between 2.5 and 5×10⁵ cells/ml. Cells are harvested after 0,1, 3, 6,12, 24,48, and 72 hours, the supernatant frozen for further investigation, cells are washed with PBS, and resuspended in 400 μl of RLT buffer (from Qiagen RNeasy Total RNA Isolation Kit) with 143 mM β-mercaptoethanol, the DNA sheared with a 20 g needle for at least 5 times and stored at −70° C.

Stimulation of cells by cigarette smoke is performed using a smoke-enriched media. 100 ml RPMI media without supplements is perfused with the cigarette smoke of 2 cigarettes. The smoke of the cigarettes is pulled into a 50 ml syringe (about 20 volumes of a 50-ml volumes per cigarette) and then perfused into the media. Afterwards, the pH of the media is adjusted to 7.4, and the media is filter sterilized through a 0.2 μm filter. Cells are resuspended in smoke-enriched media and incubated for 10 minutes at 37° C. at a density of 1×10⁶ cells/ml. Then, cells are washed twice with RPMI 1640 and seeded in flasks or 24-well plates (MonoMac6) for the times indicated above.

Total RNAs are isolated with the Qiagen RNeasy Total RNA Isolation Kit (Qiagen) according to the manufacturer's protocol. Purified RNA is used for TaqMan analysis. The expression levels of cytokines TNFα, IL-1β, IL-8, and IL-6 are measured.

Detection of Secreted Cytokines

Proteins in the supernatants of the cultured and stimulated cells are precipitated by adding trichloroacetic acid (TCA) to a final concentration of 10%. Precipitates are washed twice with 80% ethanol and pellets are resuspended in 50 mM Tris/HCl, pH 7.4, 10 mM MgCl₂, 1 mM EDTA. Protein concentration is determined via the Bradford method and 50 μg of each sample are loaded on 12% SDS polyacrylamide gels. Gels are blotted onto PVDF-membranes, blocked for 1 hour in 5% BSA in TBST, and incubated for 1 hour with commercially available antibodies against human TNFα, IL-1β, IL-8, and IL-6. After washing with TBST, blots are incubated with anti-human IgG conjugated to horseradish-peroxidase, washed again and developed with ECL chemiluminescence kit (Amersham). Intensity of the bands are visualized with BioMax X-ray films (Kodak) and quantified by densitometry.

Detection of Secreted Matrix Metalloproteases and other Proteases

The procedure is identical to the one used for cytokines. Antibodies used for Western blotting are against human MMP-1, MMP-7, MMP-9, and MMP-12.

Activity of Secreted Matrix Metalloproteases

Protease activity is determined with a fluorescent substrate. Supernatants isolated from stimulated and unstimulated cells (described above) are incubated in a total volume of 50 μl with 1 μM of the substrate (Dabcyl-Gaba-Pro-Gln-Gly-Leu-Glu (EDANS)-Ala-Lys-NH2 (Novabiochem)) for 5 minutes at room temperature. Positive controls are performed with 125 ng purified MMP-12 per reaction. Protease activity is determined by fluorometry with an excitation at 320 nm and an emission at 405 nm.

In an alternative assay to determine proteolytic activity and cell migration, a chemotaxis (Boyden) chamber is used. In the wells of the upper part of the chamber, cells (10⁵ cells per well) are plated on filters coated with an 8 μm layer of Matrigel (Becton Dickinson). In the lower compartment, chemoattractants like leukotriene B₄ (10 ng/ml), MCP-1 (10 ng/ml) are added to the media. After five days filters are removed, cells on the undersurface that have traversed the Matrigel are fixed with methanol, stained with the Diff-Quik staining kit (Dade Behring) and counted in three high power fields (400×) by light microscopy.

Chemotaxis Assay

In order to determine chemotaxis, a 48 well chemotaxis (Boyden) chamber (Neuroprobe) is used. Cells are starved for 24 hours in RPMI media without FCS. Chemoattractants (50 ng/ml IL-8, 10 ng/ml MCP-1, 10 nM lipoxin A4) and substances according to the invention are diluted in RPMI media without FCS and 30 μl are placed in the wells of the lower compartment. The upper compartment is separated from the lower compartment by a polycarbonate filter (pore size 8 μm). 50 μl of cell suspension (5×10⁴) are placed in the well of the upper compartment. The chamber is incubated for 5 hours at 37° C. in a humidified atmosphere with 5% CO₂. Then the filter is removed, cells on the upper side are scraped off, cells on the downside are fixed for 5 minutes in methanol and stained with the Diff-Quik staining set (Dade Behring). Migrated cells are counted in three high-power fields (400×) by light microscopy.

Adherence Assay

Cells are harvested, washed in PBS and resuspended (4×10⁶/ml) in PBS and 1 μM BCECF ((2′-7′-bis-(carboxyethyl)-5(6′)-carboxyfluorescein acetoxymethyl) ester, Calbiochem) and incubated for 20 minutes at 37° C. Cells are washed in PBS and resuspended (3.3×10⁶/ml) in PBS containing 0.1% BSA. 3×10⁵ cells (90 μl) are added to each well of a 96-well flat bottom plate coated with laminin (Becton Dickinson) and allowed to settle for 10 minutes. Substances according to the invention are added and plates are incubated for 20 minutes at 37° C. Cells are washed with PBS containing 0.1% BSA and adherent cells are solubilized with 100 μl of 0.025 M NaOH and 0.1% SDS. Quantification is performed by fluorescence measurement.

Phagocytosis

Cell suspensions (2.5×10⁴ cells/ml) are seeded in 6-well plates with 5 ml of U937 or THP-1 or in 24-well plates with 2 ml of MonoMac6 and incubated for 1 hour at 37° C. in a humidified atmosphere with 5% CO₂ in the presence of substances according to the invention. 40 μl of a dispersed suspension of heat-inactivated Saccharomyces boulardii (20 yeast/cell) are added to each well. Cells are incubated for three more hours, washed twice with PBS and cytocentrifuged. The cytospin preparations are stained with May-Grünwald-Giemsa and phagocytosed particles are counted by light microscopy.

### Example 3

PRK 2

Another identified gene codes for PRK2 (SEQ ID NOs:9,10). PRK2 is serine/threonine kinase related to the PKC class of protein kinases. It is a downstream effector of the small GTPases Rac and RhoA and seems to be involved in processes of cell motility (Vincent, S. and J. Settleman (1997) Mol. Cell. Biol. 17, 2247-2256).

PRK2 is consistently found downregulated (54.8%) in COPD smokers compared to healthy smokers. This is shown by “avg diff” values (Table 3). The p value for the comparisons between COPD smokers and healthy smokers is 0.02.

Table 3: Expression levels of PRK2: “avg diff” values for each patient are listed as well as mean and median values for the three groups of subjects; OS means obstructed smoker, HS healthy smoker, NS non-smoker

TABLE 3

| Subject | | Subject | | Patient | |
|---|---|---|---|---|---|
| (OS) | Avg diff | (HS) | Avg Diff | (NS) | Avg Diff |
| P01 | 234.4 | P02 | 366.2 | P48/49 | 1509.0 |
| P03 | 365.7 | P37 | 1593.9 | P50/52 | 1185.8 |
| P05 | 291.4 | P43 | 486.3 | P54/61 | 1187.3 |
| P06 | 504.5 | P56 | 1387.0 | | |
| P39 | 857.2 | P57 | 736.6 | | |
| P44 | 257.5 | P58 | 1074.0 | | |
| | | P62 | 1090.7 | | |
| Mean ± | 418.4 ± | | 962.1 ± | | 1294.0 ± |
| SD | 236.1 | | 454.8 | | 186.2 |
| Median | 328.6 | | 1074.0 | | 1187.3 |

Assays are constructed with PRK2 instead of PAK2 in a manner analogous to Example 2.9.

### Example 4

GUK1

Another gene identified is guanylate kinase 1 (GUK1; SEQ ID NOs:11, 12). Guanylate kinase 1 catalyzes the transfer of phosphate from adenosine triphosphate (ATP) to guanosine monophosphate (GMP) or dGMP. This enzyme functions in the recovery of cGMP and is, therefore, thought to regulate the supply of guanine nucleotides to signal transduction pathways (Brady, W. A. et al. (1996) J. Biol. Chem. 271,16734-16740).

GUK1 is consistently found upregulated (52%) in COPD smokers compared to healthy smokers. This is shown by “fold change” (FC) values (Table 4). The p values in two separate groups comparing COPD smokers and healthy smokers are 0.02 and 0.17.

TABLE 4
Fold change values (FC) for comparisons between obstructed
smoker and healthy smokers. On average, GUK1 is upregulated by
2.05 fold, the median is 2.05 fold.

| comp | FC | comp | FC | comp | FC | Comp | FC |
|---|---|---|---|---|---|---|---|
| 1 vs 2 | 1.9 | 5 vs 43 | 7.9 | 39 vs 57 | 1.0 | 68 vs 66 | 5.2 |
| 1 vs 37 | 6.5 | 5 vs 56 | 5.1 | 39 vs 58 | 1.3 | 68 vs 69 | 2.4 |
| 1 vs 43 | 6.8 | 5 vs 57 | 2.6 | 39 vs 62 | 1.7 | 68 vs 76 | 5.9 |
| 1 vs 56 | 4.4 | 5 vs 58 | 3.3 | 44 vs 2 | −1.5 | 68 vs 78 | 4.5 |
| 1 vs 57 | 2.2 | 5 vs 62 | 4.6 | 44 vs 37 | 2.2 | 70 vs 65 | −1.1 |
| 1 vs 58 | 2.9 | 6 vs 2 | −2.6 | 44 vs 43 | 2.3 | 70 vs 66 | 2.4 |
| 1 vs 62 | 4.0 | 6 vs 37 | 1.3 | 44 vs 56 | 1.5 | 70 vs 69 | 1.1 |
| 3 vs 2 | −1.9 | 6 vs 43 | 1.4 | 44 vs 57 | −1.3 | 70 vs 76 | 2.7 |
| 3 vs 37 | 1.7 | 6 vs 56 | −1.1 | 44 vs 58 | 1.0 | 70 vs 78 | 2.1 |
| 3 vs 43 | 1.8 | 6 vs 57 | −2.2 | 44 vs 62 | 1.4 | 71 vs 65 | 2.2 |
| 3 vs 56 | 1.2 | 6 vs 58 | −1.7 | 64 vs 65 | 1.2 | 71 vs 66 | 5.7 |
| 3 vs 57 | −1.7 | 6 vs 62 | −1.2 | 64 vs 66 | 2.4 | 71 vs 69 | 2.7 |
| 3 vs 58 | −1.3 | 39 vs 2 | −1.2 | 64 vs 69 | 1.3 | 71 vs 76 | 6.5 |
| 3 vs 62 | 1.1 | 39 vs 37 | 2.8 | 64 vs 76 | 2.4 | 71 vs 78 | 4.9 |
| 5 vs 2 | 2.2 | 39 vs 43 | 3.0 | 64 vs 78 | 1.8 | | |
| 5 vs 37 | 7.5 | 39 vs 56 | 1.9 | 68 vs 65 | 2.0 | | |

Assays are constructed with GUK1 instead of PAK2 in manner analogous to Example 2.9.

What is claimed is:

1. A method for determining whether a substance is an activator or an inhibitor of a function of a protein comprising: (a) contacting the protein with a substance to be tested, wherein the protein is a DHAM-kinase; and (b) measuring whether the function is inhibited or activated.

2. A method for determining whether a substance is an activator or an inhibitor of a function of a protein comprising: (a) contacting the protein with a substance to be tested, wherein the protein is a variant, mutant or fragment of a DHAM-kinase having a function of the corresponding DHAM-kinase; and (b) measuring whether the function is inhibited or activated.

3. The method according to claim I wherein the inhibition or activation of the desired function is measured directly.

4. The method according to claim 1 wherein the inhibition or activation of the desired function is measured indirectly.

5. The method according to claim 1 wherein the DHAM-kinase is a mammalian DHAM-kinase.

6. The method according to claim 5 wherein the DHAM-kinase is a human DHAM-kinase.

7. The method according to claim 1 wherein the method is performed using a cellular system.

8. The method according to claim 1 wherein the method is performed using a cell-free system.

9. The method according to claim 1 wherein the DHAM-kinase consists of an amino acid sequence selected from the group consisting of: SEQ ID NO:4, SEQ ID NO:10, and SEQ ID NO:12.

10. The method according to claim 9 wherein the amino acid sequence is SEQ ID NO:4.

11. The method according to claim 9 wherein the amino acid sequence is a variant, mutant or fragment of SEQ ID NO:4 having the same function of SEQ ID NO:4.

12. The method according to claim 9 wherein the amino acid sequence is SEQ ID NO:10.

13. The method according to claim 9 wherein the amino acid sequence is a variant, mutant or fragment of SEQ ID NO:10 having the same function of SEQ ID NO:10.

14. The method according to claim 9 wherein the amino acid sequence is SEQ ID NO:12.

15. The method according to claim 9 wherein the amino acid sequence is a variant, mutant or fragment of SEQ ID NO:12 having the same function of SEQ ID NO:12.

16. The method according to claim 1 wherein the function is a kinase activity.

17. The method according to claim 16 wherein the function is substrate binding.

18. The method according to claim 16 wherein the function is a specific phosphorylation of a substrate.

19. A method for determining an expression level of a DHAM-kinase comprising: (a) determining the level of the DHAM-kinase expressed in a hyperactivated macrophage; (b) determining the level of the DHAM-kinase expressed in a non-hyperactivated macrophage; and (c) comparing the level of the DHAM-kinase expressed in step (a) to the level of the DHAM-kinase expressed in step (b), wherein a difference in levels indicates a differentially expressed DHAM-kinase.

20. The method according to claim 19 wherein the macrophage is a mammalian macrophage.

21. The method according to claim 20 wherein the macrophage is a human macrophage.

22. The method acording to claim 19 wherein the difference in expression level is determined at the DHAM-kinase nucleic acid level.

23. The method acording to claim 19 wherein the difference in expression level is determined at the DHAM-kinase protein level.

24. The method according to claim 23 wherein the DHAM-kinase protein consists of an amino acid sequence selected from the group consisting of: SEQ ID NO:4, SEQ ID NO:10, and SEQ ID NO:12.

25. The method according to claim 24 wherein the amino acid sequence is SEQ ID NO:4.

26. The method according to claim 24 wherein the amino acid sequence is a variant, mutant or fragment of SEQ ID NO:4 having the same function of SEQ ID NO:4.

27. The method according to claim 24 wherein the amino acid sequence is SEQ ID NO:10.

28. The method according to claim 24 wherein the amino acid sequence is a variant, mutant or fragment of SEQ ID NO:10 having the same function of SEQ ID NO:10.

29. The method according to claim 24 wherein the amino acid sequence is SEQ ID NO:12.

30. The method according to claim 24 wherein the amino acid sequence is a variant, mutant or fragment of SEQ ID NO:12 having the same function of SEQ ID NO:12.

31. A method for diagnosing or monitoring a chronic inflammatory airway disease comprising: (a) determining the level of a DHAM-kinase expressed in a hyperactivated macrophage; (b) determining the level of the DHAM-kinase expressed in a non-hyperactivated macrophage; and (c) comparing the level of the DHAM-kinase expressed in step (a) to the level of the DHAM-kinase expressed in step (b), wherein a difference in levels indicates a differentially expressed DHAM-kinase.

32. The method acording to claim 31 wherein the difference in expression level is determined at the DHAM-kinase nucleic acid level.

33. The method acording to claim 31 wherein the difference in expression level is determined at the DHAM-kinase protein level.

34. The method according to claim 31 wherein the chronic inflammatory airway disease is selected from the group consisting of chronic bronchitis and COPD.

35. The method according to claim 31 wherein the method is performed using a macrophage or a part thereof obtainable from a site of inflammation.

36. A substance determined to be an activator or an inhibitor of a DHAM-kinase.

37. A substance determined to be an activator or an inhibitor of a DHAM-kinase according to the method of claim 1.

38. A substance for the treatment of a disease wherein the substance is an activator or an inhibitor of a DHAM-kinase.

39. The substance according to claim 38 wherein the disease is a chronic inflammatory airway disease.

40. The substance according to claim 39 wherein the chronic inflammatory airway disease is selected from the group consisting of: chronic bronchitis and COPD.

41. A pharmaceutical composition comprising at least one substance which is an activator or an inhibitor of a DHAM-kinase; and a pharmaceutical carrier.

42. A pharmaceutical composition comprising at least one substance which is determined to be an activator or an inhibitor of a DHAM-kinase according to the method of claim 1; and a pharmaceutical carrier.

43. A pharmaceutical composition comprising at least one substance which is determined to be an activator or an inhibitor of a DHAM-kinase according to the method of claim 9; and a pharmaceutical carrier.

44. A method for treating a chronic inflammatory airway disease comprising: administering to a subject in need of such treatment an effective amount of a pharmaceutical composition comprising at least one substance determined to be an activator or an inhibitor of a DHAM-kinase.

45. The method according to claim 44 for treating a mammal.

46. The method according to claim 44 for treating a human being.

47. The method according to claim 44 for treating a chronic inflammatory airway disease selected from the group consisting of chronic bronchitis and COPD.

48. A method for treating a chronic inflammatory airway disease comprising: administering to a subject in need of such treatment an effective amount of a pharmaceutical composition comprising at least one substance determined to be an activator or an inhibitor of a DHAM-kinase according to the method of claim 1.

49. A method for treating a chronic inflammatory airway disease comprising: administering to a subject in need of such treatment an effective amount of a pharmaceutical composition comprising at least one substance determined to be an activator or an inhibitor of a DHAM-kinase according to the method of claim 9.

50. A method for selectively modulating a DHAM-kinase in a macrophage, comprising administering a substance determined to be an activator or an inhibitor of a DHAM-kinase.

51. The method according to claim 50 wherein the macrophage is involved in a chronic inflammatory airway disease.

52. The method according to claim 50 wherein the chronic inflammatory airway disease is selected from the group consisting of: chronic bronchitis and COPD.

53. A method for selectively modulating a DHAM-kinase in a macrophage, comprising administering a substance determined to be an activator or an inhibitor of a DHAM-kinase according to the method of claim 1.

54. A method for selectively modulating a DHAM-kinase in a macrophage, comprising administering a substance determined to be an activator or an inhibitor of a DHAM-kinase according to the method of claim 9.
